(19) United States
(12) Patent Application Publication
(10) Pub. No.: US 2012/0010533 A1
(43) Pub. Date: Jan. 12, 2012

(54) Method for determining the correct natural head position location of references planes relative to a three-dimensional computerized image of a patient's head

(76) Inventors: G. William Arnett, Santa Barbara, CA (US); Michael J. Gunson, Santa Barbara, CA (US)

(21) Appl. No.: 13/135,501

(22) Filed: Jul. 5, 2011

Related U.S. Application Data
(63) Continuation-in-part of application No. 11/252,411, filed on Oct. 17, 2005, now abandoned.

(51) Int. Cl. A61B 5/103

(52) U.S. Cl. 600/590; 600/587

(57) ABSTRACT

A system and method for determining the correct natural head position location of reference planes, i.e., the coronal, transverse, and sagittal planes, relative to a three-dimensional computerized image of a patient's head. This method will allow medical practitioners to accurately measure various soft tissue features of the patient's face and underlying hard tissue structures, e.g., such as dentoskeletal structures, and combining such analysis with profile analysis and treatment planning for full 3D facial analysis.

### CROSS REFERENCE TO RELATED APPLICATIONS

The application is a continuation-in-part of U.S. patent application Ser. No. 11/252,411, filed Oct. 17, 2005, which claims the benefit of and the priority to 61/432,127 filed Jan. 12, 2011 and the entire contents of which are incorporated by reference herein.

### FIELD OF THE INVENTION

The invention relates generally to the field of facial measurement and analysis, and more particularly to a system and method for determining the correct natural head position location of reference planes, i.e., the coronal, transverse, and sagittal planes, relative to a three-dimensional computerized image of a patient's head. This method will allow medical practitioners to accurately measure various soft tissue features of the patient's face and underlying hard tissue structures, e.g., such as dentoskeletal structures, and combining such analysis with profile analysis and treatment planning for full 3D facial analysis.

### BACKGROUND

The study of facial aesthetics is old. Facial attractiveness, while innately recognizable by lay people and artists, has in the past been difficult to quantify. Philosophers and artists have struggled for centuries to identify the concrete structural relationships that create an aesthetically pleasing face. Relationships and proportions

It is understood that facial attractiveness can be defined by at least two main facial characteristics, namely quality of facial parts and position of facial parts. The quality of the eyes, skin, the hair, and lips alter the perception of what is beautiful. Cosmetology and the fashion industry (and to a certain extent medicine by dermatology and chemical skin peels to improve the color, tone, smoothness of the skin) are founded upon improving the quality of these features.

However, the position, shape and size of the facial features arguably have an even greater impact on facial aesthetics. For example, the position, shape and size of the cheekbones, orbital rims, nose, jaws and chin are even more major determinants of facial aesthetics, and surgeons, orthodontists, and dentists are able to affect changes to these facial features using various treatment methods.

Advancements in orthodontics and orthagnathic and craniofacial surgery have led to tremendous advancements in the available treatment for persons with congenital defects, developmental defects, victims of injury and those who simply wish to improve their facial aesthetics. It is known that changes to dentoskeletal structures, such as the teeth, cartilage and bones that underlie soft tissue will greatly influence the outward appearance of the face and head. Accordingly, for example, in orthodontics, the movement of the teeth and jaw line are important in optimizing the frontal and side profiles. While some orthodontists and orthagnathic surgeons are experienced and skillful enough to estimate what changes to the underlying hard structures will result in the most aesthetically pleasing results, for most of these professionals, standards, guidelines and specific directives are greatly preferable and result in more consistently excellent results.

The inventors have in the past developed methods of soft tissue cephalometric analysis for diagnosis and methods for cephalometric treatment planning for aesthetic correction of facial imbalance in a patient with regards to the side profile, both for undifferentiated males and females, and for differentiated male and female groups. See U.S. Pat. Nos. 5,951,498 and 6,200,278, respectively. The disclosures in U.S. Pat. Nos. 5,951,498 and 6,200,278 are incorporated herein in their entirety.

While a good profile is an essential part of good facial aesthetics, it is possible to have a good profile yet still have an overall unbalanced face if the front view has imperfections.

Currently, many medical and dental professionals use cone beam computed tomography (Cone beam CT, aka “CBCT”) in treatment planning and diagnosis for implant dentistry and facial surgery. During a CBCT scan, a scanner head of the CBCT machine rotates around a patient's head, obtaining several hundred discrete images of the patient's face and head from all around the patient's head. A CBCT scan can take up to a minute or so, and during the scan it is imperative that the patient's head remain immobilized. In order to accomplish this, while the patient is seated in the CBCT machine, his or her head will be strapped in place with a forehead strap and the patient's chin will usually be held in a chin cup. The scanning software will collect these separate images and reconstructs the images into a digital volume that is made up of three dimensional voxels (volumetric pixel) of anatomical data that can be used by specialized software to visualize and manipulate the image. Since the CBCT scan detects both hard and soft tissue, the face and head can be displayed with soft tissue skin and muscles in place, as well as provide views just depicting hard tissue, viz., bone and teeth. This features is particular useful since when using specialized treatment planning software, a practitioner can see how-changes to hard structure (e.g., teeth and bone) will change to appearance of the patient as seen at the skin level. Thus, for example, an orthodontist can determine how moving teeth will affect a patient's face from all views. Likewise, oral and maxillofacial surgeons and orthodontists interested in correcting injuries or birth defects can use treatment planning software to determine how surgical and orthodontic procedures will influence the patient's overall appearance.

In treatment planning, it is extremely important to determine the patient's natural head position. The natural head position is the position of the patient's head and face when the patient looks forward straight ahead when relaxed. In the natural head position, the head should not be tilted left or right, up or down, or side to side. In order to clinically establish the patient's natural head position, the medical practitioner will ask the patient will look into a mirror and level his/her head by moving it up and down (bringing the chin up and down) along the sagittal plane. The patient will also be asked to turn his or her head from left side to right side along the transverse plane and then look straight into the mirror into his or her own pupils. The patient will also be asked to tilt his head from shoulder to shoulder while looking into a mirror to align his head relative to the coronal plane. Carrying out these movements while the patient looks directly into the pupils normally orients the patient's head into a natural head position. The natural or postural head position thus established is based on the perspective of the patient looking in a mirror into his or her own eyes. During the process it is important the patient's joints are seated, the teeth are at first contact, and the lips are passively related. Some times, the medical professional can assist the patient in determining the natural head position. Accordingly, the natural head position is largely a patient's self-determined position, and knowing it is an important as part of treatment planning and execution so that the results are optimized. However when the patient's head is locked in place with the forehead strap and chin cup in preparation for the CBCT scan, the patient's head is rarely in the natural head position and is facing up or down, rotated on the neck, and/or tilted side to side, with the result that when the scan is taken, the head is not a natural head position. Therefore, the three-dimensional computerized image of the patient's face and head thus scanned will not be correlated to either the patient's “natural head position” and the positions of the coronal, transverse, or sagittal planes will not be knowable to the practitioner viewing and needing to work with the 3D computerized image of the patient's head and face. Thus, the practitioner who is using the specialized imaging software will be at a great disadvantage when using the CBCT scanned 3D image of the patient's head and face in treatment planning and execution.

Indeed, with respect to all procedures, it is important that an accurate frontal and profile facial analysis be available to ensure that the frontal and profile views of a patient's face are aesthetically optimized. Moreover, by correctly identifying the positions of the coronal, transverse, or sagittal planes in the 3-D computerized mode, a person's entire facial balance can be optimized from all views (3D), resulting in an aesthetically pleasing facial appearance from any angle.

There accordingly remains a need for a method for determining the correct natural head position locations of reference planes, i.e., the coronal, transverse, and sagittal planes, relative to a three-dimensional computerized image of a patient's head.

### SUMMARY OF THE INVENTION

One object of the invention is to provide a method to correlate a profile view of a patient's face in the natural head position with a 3D computerized view of the patient head and face to aid in an analysis that focuses on soft tissue landmarks as opposed primarily to hard tissue to achieve consistently excellent outcomes in dental, orthodontic and surgical correction of facial imbalance in frontal view and in frontal plus profile view so that the face and head may be studied in 3D from a variety of angles.

Another object of the invention is to provide health care professionals with a discrete treatment plan to guide in precisely what action must be taken to improve facial balance whether in a frontal view, or frontal and profile views and any other views thereof.

The diagnosis and treatment normal values of the current invention will be carried out by establishing normal size, distance, and angle ranges of various facial landmarks of aesthetically pleasing faces from a population group, and having a patient place his or her head in a natural head position, and correcting that position if necessary, and establishing an anatomical position of the head to locate the position of the true vertical line (through which the coronal plane passes), and correlating the position with the true vertical line with soft tissue structures of the patient's head and face. The known position of the patient's head in natural head position will then be used to determine the correct position of the coronal plane in 3D image of the patient's head and face from the CBCT scan. Thereafter, the three perpendicular anatomical planes, namely, the sagittal, transverse and coronal planes will be correlated, and measuring soft and hard tissue landmarks to these anatomical planes to define skeletal and/or soft tissue measurements for the face, and then making orthodontic, dental, and surgical corrections to improve the facial balance and aesthetics as necessary.

In one embodiment, the invention provides a system and method for determining the correct natural head position locations of the coronal, transverse, and sagittal planes, relative to a three-dimensional computerized image of a patient's head, comprising, in not necessary the following order: having a patient place his or her head in a natural head position; clinically determining the position of the coronal plane relative to a profile view of the patient's head; taking a multiple images of a patient's head and face in order to generate a 3-D computerized image of the patient's face and head and displaying the image in software; transferring the determined position of the coronal plane to the computerized image of the patient's head; clinically determining the position of the transverse plane relative to a front view of a patient; transferring the determined position of the transverse plane to a computerized image of the patient's head; clinically determining the position of the anterior origin of the sagittal plane relative to a front view of a patient; transferring the determined anterior origin position of the transverse plane to a computerized image of the patient's head; clinically determining the position of the posterior origin of the sagittal plane relative to a front view of a patient; and transferring the determined posterior origin position of the transverse plane to a computerized image of the patient's head.

### BRIEF DESCRIPTION OF THE DRAWINGS

FIG. 1 is a diagrammatic front perspective view showing a human head and the perpendicular sagittal SP, coronal CP and transverse TP or the coplanar anatomical planes ASP, ACP, ATP.

FIG. 2 diagrammatic side profile view of a patient with his head in the natural head position and a practitioner using a frame grid tool with guide lines in order to determine the position of the true vertical line.

FIG. 3 is a diagrammatic frontal facial view showing the sagittal plane SP or anatomical sagittal plane ASP passing through the midline of the face and landmarks on the face and facial outline landmarks.

FIG. 4 is a diagrammatic frontal facial view showing the sagittal plane SP or anatomical sagittal plane ASP, the transverse plane TP or anatomical transverse plane through Na′ ATP-Na′ and various measurement landmarks and lines for the middle and lower ⅓rds of the face measured perpendicular to ASP and parallel to ATP-Na′.

FIG. 5 is a diagrammatic frontal facial view showing the sagittal plane SP or anatomical sagittal plane ASP and the transverse plane TP or anatomical transverse plane passing through nasion ATP-Na′, with levels drawn though the eyes, upper canine teeth, lower canine teeth, inferior border of the lower jaw, and chin, with the cheekbone contour also illustrated. Also shown are the intercanthal distance ICD and the alar base width ABW.

FIG. 6 is a diagrammatic right side facial profile view showing the position of the coronal plane CP or anatomical coronal plane drawn through subnasale ACP-Sn and facial profile landmarks.

FIG. 7 is a diagrammatic frontal facial view showing width measures of the facial outlines.

FIG. 8 is diagrammatic frontal facial view showing facial symmetry measurements.

### DETAILED DESCRIPTION OF THE INVENTION

In the invention, the frontal head posture and measurements will be used in conjunction with the profile postural head position and measurements in order to create a 3-D model of the face and head.

A frontal and profile head position images will be taken with conventional CT scans, cone beam computed tomography (CBCT), 3D photography, 2D photography, scattered light photography, laser scanning, profile and frontal cephalometrics, ultrasound and magnetic resonance imaging, or any other imaging technique that presently exists or in the future may be developed.

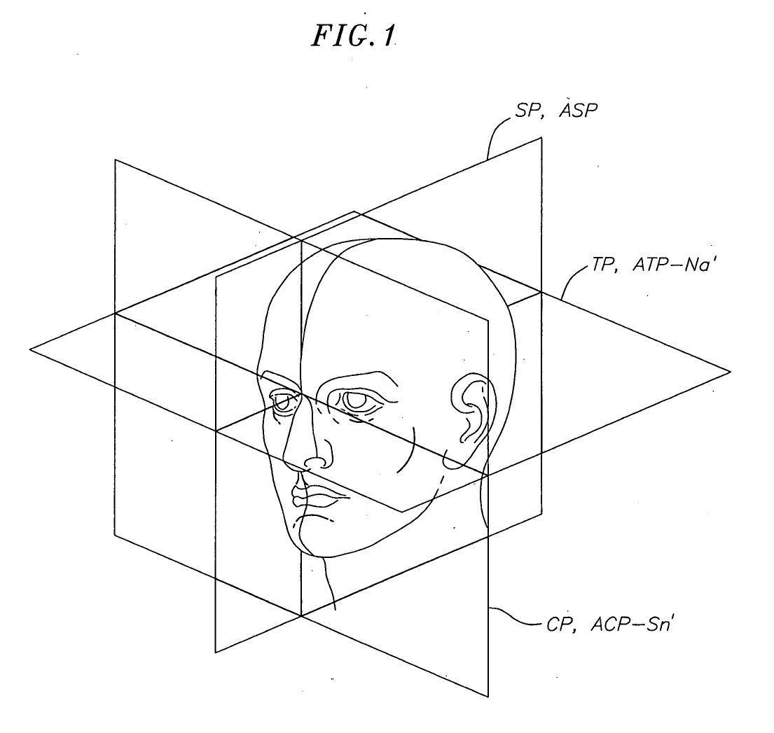

In order to position the head in the proper anatomical position, it is necessary to properly orient the head with respect to three perpendicular anatomical reference planes, namely the sagittal plane SP, the transverse plane TP, and coronal plane CP. These three anatomical planes of references are shown in FIG. 1 with reference to a diagrammatic head and face.

The sagittal plane SP is perpendicular to the floor, runs front to back and up and down. The anatomical sagittal plane is generally utilized to measure the width of the face. The anatomical sagittal plane ASP is a vertical plane that passes through the centerline of the patient's face from front to back, top to bottom, and divides it symmetrically. If the head is properly positioned and balanced, the sagittal plane will pass longitudinally through the centerline of the nose. If a patient tilts his/her chin up or down along the sagittal plane, this will not affect the distances of landmarks to the sagittal plane. However, if the head is tilted to the left or right shoulder, or the head is turned left or right, then the landmarks will move out of alignment with the sagittal plane.

The transverse (or axial) plane TP is a plane parallel to the ground and goes from left to right, front to back. The anatomical transverse plane is generally used to measure the height of the face on the left and right sides. By way of example, when a facially balanced and symmetrical head is in a perfect transverse head position, symmetrical horizontal landmarks on the face and head (e.g., the pupils of the eyes) will lie on the same transverse plane. Turning of the head left to right will not cause the horizontal landmarks on the face and head to be taken out of the transverse plane. In contrast, if the head is tilted to the left or right shoulder, this will affect the distances of the landmarks to the transverse plane TP. If the chin is brought up or down, perpendicularly to the transverse plane TP, this will also affect the distances of the symmetrical landmarks to the transverse plane TP, but equally to both sides.

The coronal plane CP is the vertical plane of reference that is parallel to the front of the patient's face (up and down) and follows from left to right. The coronal plane (CP) is used to measure the projection of the left and right sides, and midline of the face. The coronal plane will pass through the true vertical line (TVL). When the patient's head is in the natural head position, the TVL passes through subnasale Sn (a point just under the patient's nose) and is vertical to the floor. The TVL will also typically pass through a point 6-11 mm (8.25±2.5 mm) in front of soft tissue glabella G′ (the prominent most point of the forehead), (anterior glabella point AG′P). If the patient's face is facially balanced and properly oriented, the same landmark on the right and left side of the face will be equal distance from the coronal plan CP (see FIG. 6). Regardless of whether the face is facially balanced, tilting of the head from side to side will not affect the distances of landmarks to the coronal plane. However, if the head is turned axially from side to side or the chin is brought up or down, this will affect the distances of the landmarks to the coronal plane.

Since the face is ideally laterally symmetrical viewed from the frontal view, the distances of symmetrically located anatomical features, e.g., the two pupils of eye, from the sagittal plane and from the transverse plane should be the same in a symmetrical and balanced face.

Thus, it can be appreciated that the proper position of the head in the three planes of reference is important in order to create the proper 3-D diagnosis and treatment plan of the head.

In order to position the head in the correct orientation, the following exemplary technique may be used. This exemplary technique is representative of the possible techniques that can be used and is not meant to be limiting. Proper posturing is imperative to insure the reliability of any analysis and treatment based on the captured image.

First, the patient will be asked to look straight ahead into a mirror at his or her pupils and establish a natural head position. In order to do this, the patient will look into a mirror and level his/her head by moving it up and down (bringing the chin up and down) along the sagittal plane. The patient will also turn his or her head from left side to right side along the transverse plane and then look straight into the mirror into his or her own pupils. The patient will also tilt his head from shoulder to shoulder while looking into a mirror to align his head relative to the coronal plane. Carrying out these movements while the patient looks directly into the pupils orients the patient's head into a natural head position.

The natural or postural head position thus established is based on the perspective of the patient looking in a mirror into his or her own eyes. During the process it is important the patients joints are seated, the teeth are at first contact, and the lips are passively related. The postural head position will thus be oriented in 3-D space relative to the perpendicular sagittal, transverse and coronal planes. The straight ahead vertical and horizontal planes thus established by the patient from his or her own perspective are referred to herein as the “postural sagittal plane PSP”, the “postural transverse plane PTP” and the “postural coronal plane PCP”. However, the postural sagittal plane, the postural transverse plane and the postural coronal plane may or may not in fact be anatomically accurate and could be out of alignment with one or more of the anatomical sagittal, transverse and coronal planes. The postural head position may be altered by postural habits which the patient has acquired or imaging techniques and equipment may make postural head position impossible for the patient to achieve during image procurement.

If the practitioner determines that the patient's head position needs adjustment, adjustments can be made at this stage.

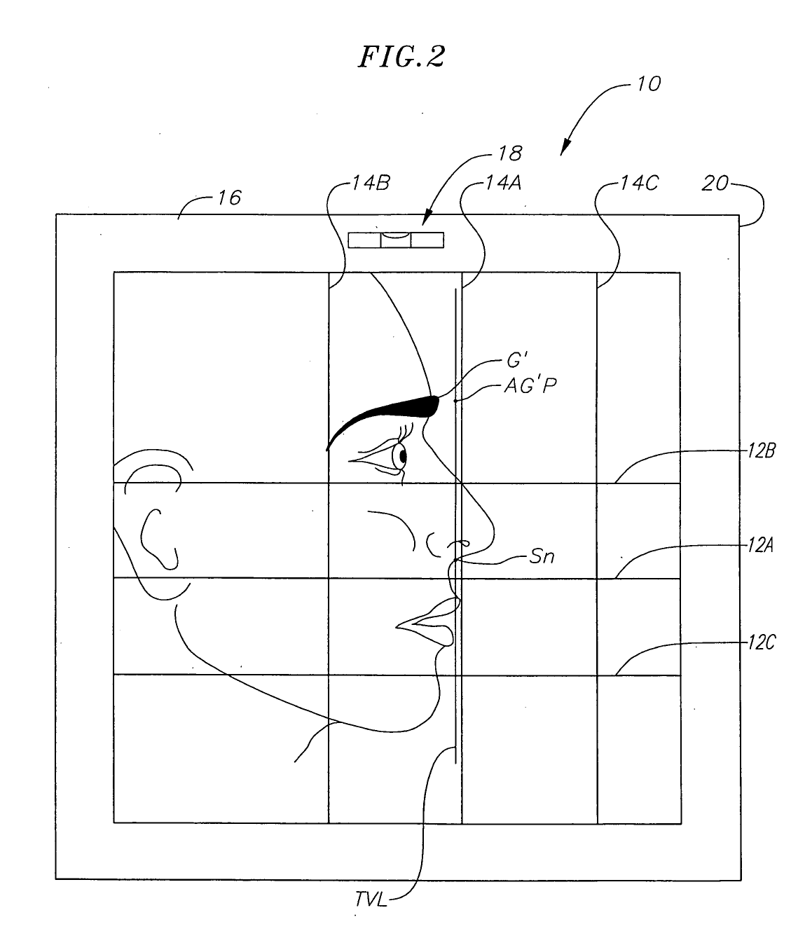

After the head is determined to be in the natural head position, as shown in FIG. 2, the practitioner will use a frame grid 10 to determine the position of the true vertical line (TVL), which also corresponds to an end view of the coronal plane CP. The frame grid 10 has at least two straight lines 12A and 14B that are perpendicular to each other. Preferably, the frame grid 10 will include several parallel horizontal lines 12A, 12B, 12C, and several parallel vertical lines 14A, 14B, 14C. The frame grid 10 can include a perimeter frame 16, with the horizontal lines 12A, 12B, 12C and vertical lines 14A, 14B, 14C comprising thin wires. Alternatively, printed lines can be placed on a piece of transparent material in lieu of using wires. A bubble level 18 can be included in the frame 16 so that the practitioner will know when the frame is being held with its wires being oriented vertically and horizontally. In lieu of include a bubble level 18, the practitioner can align an edge, e.g., 20 of the perimeter frame 16 with an object known to be perfectly vertical, such as the edge of a door, lines on a wall, etc.

In use of the frame grid 10, the practitioner will take the frame grid, hold it up in front of the patient's face so that the vertical line 14A passes through subnasale Sn (a point just under the patient's nose). The practitioner will then adjust the frame grid 10 so that the vertical line 14A is perfectly vertical (such as by tilting the frame grid 10 to align the bubble level or align the edge 20 with a perfectly vertical line on wall (not shown). In FIG. 2, vertical line 14A and horizontal line 12A are shown slightly offset from passing through subnasale Sn but the vertical line 14A is still parallel to TVL. Once the frame grid 10 is in the correct position position, the position of the TVL (the end view of the coronal plane) is established. The TVL will typically pass through a point 6-11 mm (e.g., 8.5 mm±2.5 mm) in front of soft tissue glabella G′ (the prominent most point of the forehead), namely, the anterior glabella point AG′P. The practitioner will measure the distance from soft tissue glabella G′ to the TVL to determine the precise measurement of the patient being examined. This number will be recorded and used in the next phase of the analysis.

In the next phase of the analysis, a CBCT scan will be taken of the patient's face and head. As previously noted, the patient's head will generally not be in the natural head position when it is scanned. However, the data gathered from the step of establishing the position of the TVL (end view of the coronal plane) from viewing the patient's profile will come into play at this stage.

Once the CBCT scan of the face and head is taken and the data is uploaded into the 3D analysis software, a profile view will be used that shows the patient skin and other soft tissue. The practitioner will draw in a vertical line in the software that passes through subnasale (Sn). The distance of the vertical line in the software from the soft tissue glabella G′ (the prominent most point of the forehead) to the vertical line will be measured, and the profile image of the head will be rotated around the point the vertical line passes through subnasale (Sn) until the distance from glabella G′ to the vertical line equals the distance determined from using the frame grid 10. Once this is accomplished, the practitioner will know the position of the TVL which also corresponds to the position of the coronal plane since the coronal plane will place through the TVL. Alternatively, the practitioner can place a point off of glabella G′ that is the measured distance determined from using the frame grid from the glabella G′ to the TVL. The practitioner can then rotated the profile image of the patient's head until the vertical line intersects subnasale (Sn) and glabella G. Once this is done, this again will be the position of the TVL and the position of the coronal plane.

In next phase, the transverse or axial plane will be located. This can be done using a clinical measure, such as the level of the upper canine teeth, which are generally level, but may have some disparity, such as one being lower than the other, e.g., the level of the upper right canine tip being 2.00 mm lower than the upper left canine tip. The practitioner will then call up a front CBCT view of the patient's skull and view hard tissue. The practitioner will draw a horizontal line in the software and see how it aligns with the determined clinical measurements of the upper canine teeth. If the drawn in horizontal line does not property align with the upper canines as clinically determined, then the image of the head in the software will be tilted to the left or right until the drawing of the horizontal line is properly aligned with the image of the skull, e.g., with the level of the upper right canine tip being 2.00 mm lower than the upper left canine tip relative to the horizontal line. When this is accomplished, the position of the transverse plane will be accurately located. However, as a secondary plane level check, the practitioner can compare the determined position of the transverse plane with other anatomical landmarks. For example, with soft tissue being shown, the practitioner can see how the determined transverse plane is positioned relative to the corners of the eyes. In lieu of soft tissue, with the hard tissue being shown, the practitioner can check to see how the determined level of the transverse plane aligns with the inferior border of the mandible, the orbital rims, the frontozygomatic sutures, and/or the inferior orbital rims. If these levels appear to be off, then the practitioner can go back and make sure that the position of the transverse plane through the tips of the upper canines was not erroneous, and make any necessary adjustments. Once this is complete, the position of the transverse plane relative to the image of the head and skull in the CBCT software will be determined, and will be perpendicular to the coronal plane. The last plane to determine is the position of the sagittal plane, which is described below.

To determine the sagittal plane, the practitioner will first need to determine the anterior origin of the sagittal plane (the front edge of the sagittal plan). This can be done by taking a clinical measurement to determine the upper midpoint of the upper lip. The practitioner will then transfer this upper midline clinical measurement to a front view computerized image of the patient's head in the 3D software. A vertical line will be drawn through the upper midpoint of the upper lip. If the vertical line does not seem to pass through proper landmarks on the face, such as the nasal bridge and the nose, then the image of the face can be shifted to the left or right relative to the vertical line as required. These steps will determine the anterior origin of the sagittal plane, but does not account for the yaw of the sagittal plane as it passes through the patient's head between the eyes and out the back of the head. In order to determine the posterior origin of the sagittal plane, the practitioner, can use his/her clinical judgment based on the left to right symmetry of hard tissue landmarks, such as the maxillary second molars. For example, the practitioner can ask the patient to open his or her mouth and observe the position of the second maxillary molars in the mouth and determine if the maxillary arch and the second molars are symmetrically aligned in the mouth. If it is determined that there is some misalignment, the practitioner will collect such data. Next, the practitioner will transfer maxillary second molar measurements to the 3D software, and using an upwardly looking software view through the head into the mouth, the image of the head can be rotated side to side along the anterior origin of the sagittal plane so that the thusly determined sagittal plane is correctly positioned relative to second maxillary molars in the 3D image of the face and head. As a second check of the posterior sagittal plane, the position of the sagittal plane relative to lateral orbital rims, and mandibular angles can be made.

When all three of the coronal, transverse, and sagittal planes are located with respect to the 3D image of the face and head, medical and dental professionals will positively know how the computerized 3D image relates to the patient's actual face and head in the natural head position, and treatment planning can be carried out with a great level of assurance.

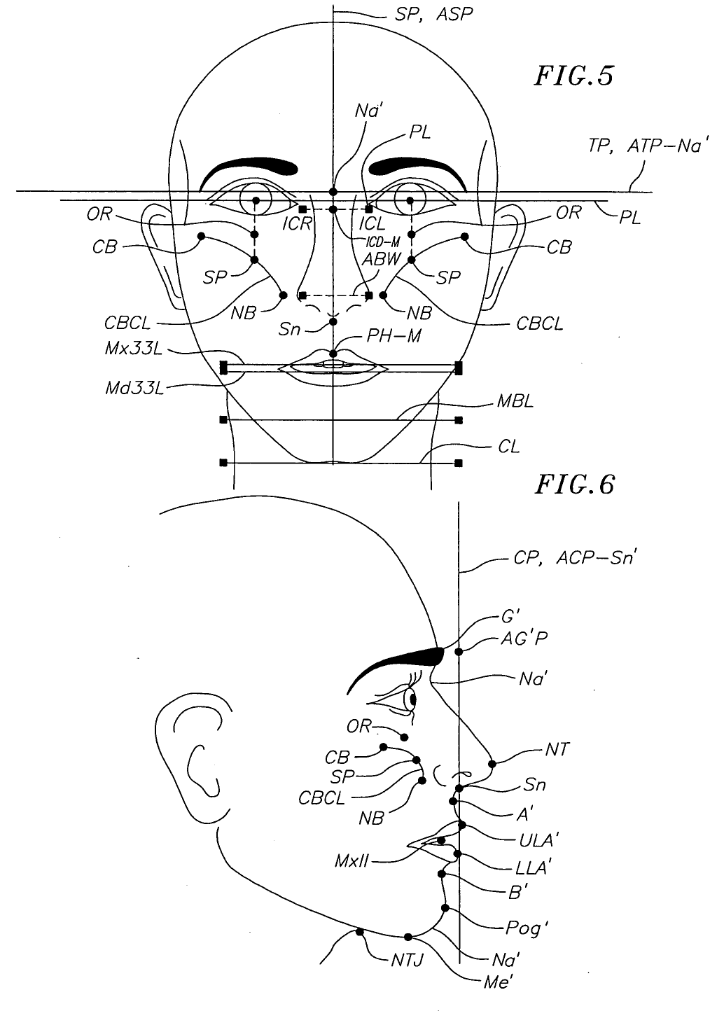

As shown in FIGS. 3 and 5, the coordinates of the anatomical sagittal plane ASP passes through the midpoint of the intercanthal distance ICD-M and the midpoint of the upper lip as defined by the midline of the philtrum PH-M, with the ASP (ICD-M to PH-M) extending above the hair line to below the soft tissue menton Me′. The anatomical sagittal plane ASP is perpendicular to the floor and is used to measure hard and soft tissue structures of the face as described below. Of course, if the postural sagittal plane passes through the same points as what would be the anatomical sagittal plane, then no correction is required. Inasmuch as the above identified coordinates (ICD-M to PH-M) are used to position the landmarks of the anatomic sagittal plane ASP (which will correspond to the postural sagittal vertical plane in a face that is correctly postured by the patient and/or practitioner in the mirror and is naturally correctly balanced), it is possible to entirely skip over the steps the patient undertakes for self-evaluation of the postural or sagittal plane and go directly to the steps undertaken by the medical professional to determine the anatomical sagittal plane ASP.

The patient starts the process in the mirror. This is verified or corrected by the practitioner as necessary and the anatomic sagittal plane ASP can then be applied after imaging is procured. (This process is done this way because some patients are not capable of displaying natural head position because of head orientation habits and imaging equipment and techniques may make it difficult if not impossible for the patient to assume the natural head position.)

Tilting of the head to the left or right shoulder is corrected by constructing the anatomical sagittal plane (ASP). Rotation of the sagittal plane about the axis of the head to the left or right is corrected by equalizing the distance from the left and right pupils to the sagittal plane.

Likewise, as shown in FIG. 6, the anatomical coronal plane ACP can be derived. The plane is constructed through subnasale Sn and a point 6-11 mm (8.25 mm±2.5 mm) in front of soft tissue glabella G′ (anterior glabella point AG′P). The ACP is perpendicular to the floor, perpendicular to the anatomical sagittal plane ASP and is the anatomical correction for profile postural head position. The exact position of anterior glabella point AG′P is determined by clinical examination of the patient while looking in straight ahead gaze into the mirror without interference by image acquisition equipment. Head position correction by the doctor or operator (corrected postural position) may be necessary to establish the position of anterior glabella point AG′P location. When midface retrusion is diagnosed, the subnasale point is moved 1-3 mm anterior. Midface retrusion is defined by a long nose, deficient alar base, poor incisor upper lip support, upright upper lip and/or thick upper lip.

After orientation of the head via the anatomic sagittal plane and the anatomic coronal plane the transverse plane is established as a perpendicular to the other two planes. The three anatomical planes (if correction is required), are used in several ways. Herein below, the term “anatomical planes” will be sometimes used to refer to the postural, doctor or operator corrected position or to the anatomical planes. First, the anatomical planes are used to measure the width equality of the left and right sides of the face at a variety of levels (i.e., zygomatic arches or angles of the mandible, etc.) Second, the anatomical planes can be used to measure the distance to the same landmarks (i.e., pupils of the eyes, angles of the mandible, upper and lower canine teeth) on both the left and right sides of the face to determine if these landmarks are on the same level to the transverse plane and/or are perpendicular to the anatomical sagittal plane, or in other words, used to make a measurement of vertical symmetry. Third, the anatomical planes can be used to orient the face for measurement of the height and width and proportionality of those distances. Fourth, the anatomical planes can be used to orient the face for vertical measurement of the face (i.e., upper ⅓ height, upper incisor exposure, etc.) A fifth use of the anatomical planes is to orient the face for a variety of structural measurements (i.e., alar base width [soft tissue], maxillary intercanine width [hard tissue], etc.) As a sixth use, the anatomical planes can be used to orient the face for shape and contour measurements (i.e., cheekbone contour, bridge of nose line).

Key landmarks of the face and head are measured to the anatomical planes. These include some or all of the following:

a. Any hard or soft tissue structures selected by the examining medical professional or other operator. These can include, for example, soft tissue or skeletal angles of the mandible.

b. Vertical structures, for example middle ⅓ of face, upper and lower lip heights.

c. outline structures, i.e., zygomatic arch width, mandibular body width.

d. Structure levels, i.e., eyes, maxillary canines, chin.

e. Midlines structures, i.e., tip of nose, upper incisor midline, and chin.

f. Left and right sides of face, i.e., cheekbone height of contour, angles of mandible.

g. Shape and contour symmetry, i.e., base of nose width, cheekbone contour.

The anatomical planes are also used to orient the face for measurement of any internal structures, such as the base of nose, bony piriform rim, zygomatic buttresses etc.) There are innumerable possibilities which can be measured. Specific measurements would be chosen by the practitioner depending upon specialty.

To facilitate the above measurements, frontal soft tissue and hard tissue facial analysis landmarks are identified using the postural, doctor or operator corrected, or anatomical planes. FIGS. 3-8 refer to some sample measurements which can be used for diagnosis and treatment planning in the area of orthodontics and orthagnathic procedures. A craniofacial surgeon may wish to use different and/or additional landmarks to measure based upon his needs. A table of measurements can be utilized and additional measurements can be taken depending upon the practitioners specific needs.

Referring again to FIG. 3, there is shown a diagrammatic frontal facial view with the sagittal plane SP and the anatomical sagittal plane ASP, midline measurement structures, outline structures, and right and left structures shown. These midline landmarks include the inner canthal distance midline ICD-M, nasal tip NT, philtrum midline PH-M, the midline between the two central maxillary incisors Mx11, the midline between the two central mandible incisors Md11, and the chin midline CM. The anatomical sagittal plane is drawn through ICD-M and PH-M. Outline landmarks include left and right chin Cn, angle points AP, mandibular body points MB, and zygomatic arches ZA.

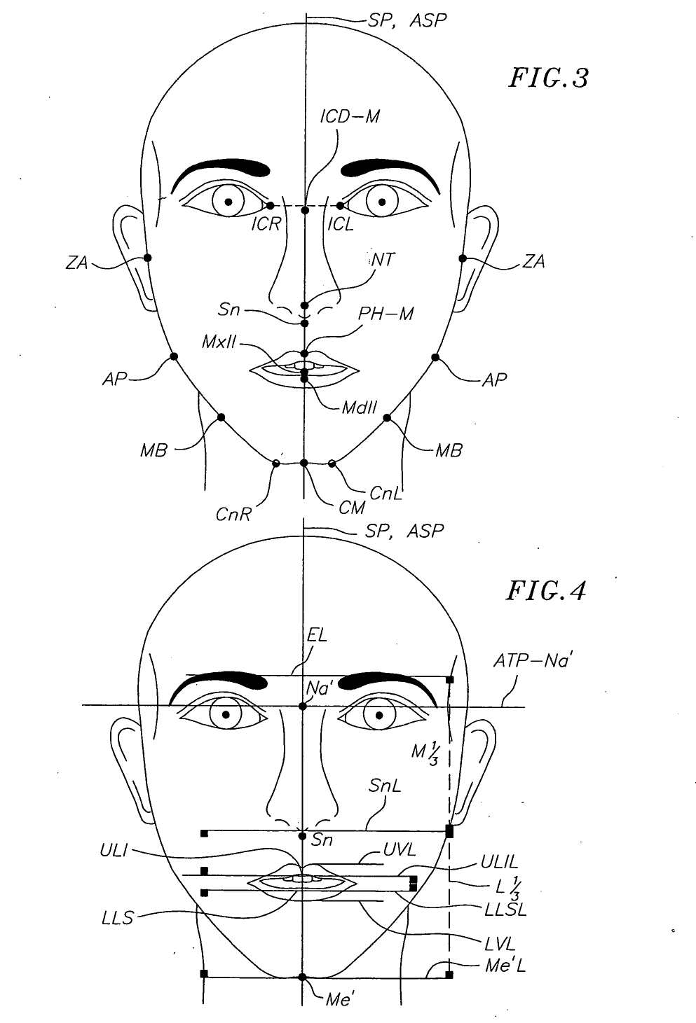

FIG. 4 is a diagrammatic frontal facial view with vertical landmarks and measurements being shown. The measurements are measured parallel to the ATP-Na′line. All measurements are taken with the head in the anatomical aligned position relative to the sagittal, transverse and coronal planes. Additional landmarks and horizontal one-third lines are shown, namely a horizontal line EL at the eyebrow level and a horizontal line SnL at subnasale level which define the middle third (M⅓), and the horizontal line SnL at the subnasale level and a horizontal line Me′L through soft tissue menton point Me′ which defines the lower third (L⅓). Additional horizontal lines are shown, namely a horizontal line ULIL through upper lip inferior point (ULI), a horizontal line LLSL through the point lower lip superior (LLS), a horizontal line UVL at the junction of the upper vermillion and skin, a horizontal line LVL at the junction of the lower lip vermillion and the skin. The interlabial gap is defined by the gap between the horizontal line ULIL and the horizontal line LLSL. The upper lip length is SnL to ULIL and the lower lip length is LLIL to Me′L. The upper vermillion height is UVL to ULIL while the lower lip vermillion is LLIL to LVL.

FIG. 5 also shows additional landmarks and various measurements. The sagittal plane (SP) and it co-planar anatomical sagittal (ASP), are depicted. All measurements are taken with the head in the anatomical aligned position relative to the sagittal, transverse and coronal planes. In FIG. 5, levels are depicted for the pupils of the eye PL, maxillary canines Mx33L, mandibular canines Md33L, body of the mandible MBL, and bottom of the chin CL. Additionally, the cheekbone contour, intercanthal distance ICD, and alar base width ABW are depicted. “Level” is when structures are on a line that is perpendicular to the anatomical sagittal plane ASP. For example, the pupils are level when they both are on a line perpendicular to the anatomical sagittal plane. Mirrored structures, which are otherwise supposed to be symmetrical located, are canted when they do not fall on a line perpendicular to the ASP. (i.e., maxillary canine on left down by 2 mm relative to the right canine).

In FIG. 5 it should be noted that the pupil line PL often is not perpendicular to the anatomical sagittal plane ASP. Thus, the pupil line PL should not be used as a base line to level other facial structures to (i.e., Mx33 level). Cheekbone contour lines CBCL can be drawn through the cheekbone CB, subpupil SP and nasal base NB landmarks on the left and right sides of the face. When these lines are flat, a cheekbone augmentation is indicated.

As an additional method to measure vertical symmetry and facial heights the anatomical transverse plane (ATP) is drawn through soft tissue nasion Na′ ATP-Na′ and is drawn perpendicular to the anatomical coronal plane ACP and the anatomic sagittal plane ASP. The transverse plane TP and anatomical transverse plane through nasion (ATP-Na′) are depicted in FIG. 5. The anatomical transverse plane through Na′ ATP-Na′ is used as a reference plane to measure the vertical position of hard and soft tissue structures. Measurements from the anatomical transverse plane through Na′ATP-Na′ to the same landmark on the left and right side of the face indicate vertical symmetry of facial structures (i.e., maxillary molar mesial buccal cusp tips, orbital rims, left and right chin). If the practitioner desires, the anatomical transverse plane (ATP) can be drawn at any level and does not have to be through soft tissue Na′.

FIG. 6 is a diagrammatic right side facial profile view depicting profile measurements measured concurrently to the frontal examination, with the anatomical coronal plane ACP shown passing through subnasale Sn. All measurements are taken with the head in the anatomical aligned position relative to the sagittal, transverse and coronal planes. The anatomical coronal plane ACP can be determined as set forth in U.S. Pat. Nos. 5,951,498 (for an undifferentiated group of males and females) and 6,200,278 (for differentiated groups of males and females) for determining the true vertical line TVL described therein, with the anatomical sagittal plane ASP passing through the true vertical line TVL as described therein. The anatomical transverse plane (ATP) is by definition perpendicular to the anatomical transverse and sagittal planes and the TVL described therein. The contents of U.S. Pat. Nos. 5,951,498 and 6,200,278 are incorporated herein by reference. Other points on the facial profile, such as the soft tissue glabella G′, anterior glabella point AG′P, nasal tip NT, soft tissue A point A′, upper lip anterior ULA′, maxillary incisor Mx11, lower lip anterior LLA′, soft tissue B point B′, soft tissue pogonion Pog′, soft tissue menton Me′ and neck throat junction NTJ are shown. The cheekbone contour line CBCL on the right side of the face is shown drawn through the cheekbone CB, subpupil SP and nasal base NB landmarks. Together, measurements from the anatomical sagittal plane ASP and the anatomical coronal plane ACP will allow mapping of these points in 3-D space. In the method of the invention, a left side facial profile view should be taken, as the left and right sides of the faces are often not perfectly symmetrical, and horizontal distances from the landmarks, e.g., the cheekbone CB, subpupil SP and nasal base NB landmarks on the left and right sides of the face can differ as measured to the ACP.

When desired or necessary an appropriate facial landmark identification object or mark can be applied to the skin prior to imaging. The type of identification landmark may be different depending on the type of imaging being used (i.e., cone beam CT, 2-D or 3-D photography, scattered light photography, laser scanning, ultrasound, magnetic resonance imaging and cephalometric x-ray). For example, objects as simple as pen markings or small (e.g., round) adhesive markers which do not distort the imaging modality being used (i.e., metal distorts CT imaging) can be used.

The anatomical sagittal plane ASP, the anatomical coronal plane ACP, and the anatomical transverse plane ATP, as defined above, are perpendicular. By perpendicularly aligning the anatomical sagittal plane ASP (ICD-M to PH-M), and the anatomical coronal plane ACP through subnasale (ACP-Sn), as well as the anatomical transverse plane ATP through Nasion (ATP-Na′) will establish a 3-D reference frame from which all landmarks (both soft tissue and hard tissue) can be identified and measured, which in turn are used to mathematically measure key features of the face and head. These figures will allow an accurate 3D plot of facial landmarks and features to be determined, and therefore will guide health care professionals in treatment planning and execution of facial improvement procedures, such as orthodontics, orthagnathic and craniofacial surgery. The assignment of the planes to specific landmarks such as the anatomical transverse plane at nasion ATP-Na′ may be altered at the practitioner's discretion if deemed desirable.

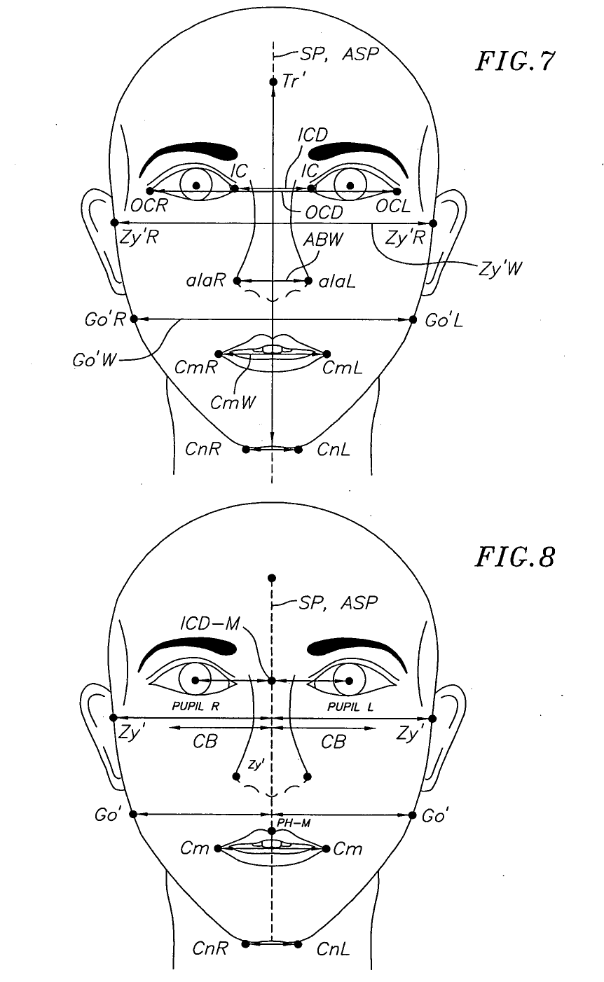

FIG. 7 is a diagrammatic frontal facial view showing some facial outlines and midlines. Selective distances are shown including the distance between the midline hair line Tr′ and menton Me′, the intercanthus distance ICD between the medial edges of the left and right eyes, and the outercanthus distance OCD between the lateral edges of the left and right eyes OCL and OCR, respectively, the alar base width ABW that extends between the alar base crease left alaL and alar base crease right alaR, chin width CnW and lastly the commissure width CmW that extends between the commissure left CmL and CmR. Also shown are the left and right zygomatic arches Zy′L and Zy′R, the gonion left and gonion right, Go′L and Go′R, respectively, and the chin left CnL and CnR.

FIG. 8 is diagrammatic frontal facial view showing facial symmetry. The anatomical sagittal plane ASP passes through the inner canthal midline ICD-M and the philtrum midline PH-M. From the anatomical sagittal plane ASP, lateral distances to certain structures are measured to determine symmetry. These include, for example, the distances from the zygomatic arch left Zy′L and the zygomatic arch right Zy′R to the anatomical sagittal plane ASP, the distances from the cheek bone left CB′L and cheek bone right CB′R to the anatomical sagittal plane ASP, and distances from the gonion left Go′L and gonion right Go′R to the anatomical sagittal plane ASP.

Table 1 shows various measures from a frontal examination.

TABLE 1
PHOTO FRONTAL EXAMINATION

| MEASUREMENTS | Normal |
|---|---|
| Midlines: (frontal View) | |
| ICD-M (inner canthal distance | All on ICD-M to PH-M line |
| midline) | |
| NT (nasal tip) | |
| PH-M (philtrum midline) | |
| Mx11 (incisor midline) | |
| Md11 (incisor midline) | |
| CM (chin midline) | |
| Symmetry: (frontal view) | |
| Zy′R (zygomatic arch right) | Measured to ICD-M to PH-M line |
| Zy′L (zygomatic arch left) | Equal right and left |
| Go′R (Md angle right) | |
| Go′L (Md angle left) | |
| Me′R (chin right) | |
| Me′L (chin left) | |
| CBR (cheekbone right) | |
| CBL (cheekbone left) | |
| Levels: (frontal view) | |
| pupil L-pupil R (interpupil) | All parallel to ATP-Na′ or |
| Mx3L-Mx3R (Mx intercanine) | perpendicular to ASP |
| Md3L-Md3R (Md intercanine) | |
| CmL-CmR (intercommissure) | |
| Md3L-Md3R (intercanine) | |
| Widths: (frontal view) | |
| pupil L-pupil R (globe width) | Measured parallel to ASP-Na′ |
| ICL-ICR (inner canthal width) | or perpendicular to ASP |
| OCL-OCR (orbital width) | |
| alaL-alaR (nasal base width) | |
| CmL-CmR (mouth width) | |
| Me′L-Me′R (chin width) | |
| Outline: | |
| Tr′-Me′M (facial height) | |
| Zy′L-Zy′R (midface width) | |
| Go′L-Go′R (lower face width) | |

Table 1 is just exemplary of some of the measurements that can be taken and is not intended to be limiting in any way.

Although embodiments of the present invention have been described in detail hereinabove in connection with certain exemplary embodiments, it should be understood that the invention is not limited to the disclosed exemplary embodiments, but, on the contrary is intended to cover various modifications and/or equivalent arrangements included within the spirit and scope of the present invention, as defined in the appended claims.

What is claimed is:

1. A system and method for determining the correct natural head position locations of the coronal, transverse, and sagittal planes, relative to a three-dimensional computerized image of a patient's head, comprising, in not necessary the following order:
having a patient place his or her head in a natural head position;
clinically determining the position of the coronal plane relative to a profile view of the patient's head;
taking a multiple images of a patient's head and face in order to generate a 3-D computerized image of the patient's face and head and displaying the image in software;
transferring the determined position of the coronal plane to the computerized image of the patient's head;
clinically determining the position of the transverse plane relative to a front view of a patient;
transferring the determined position of the transverse plane to a computerized image of the patient's head;
clinically determining the position of the anterior origin of the sagittal plane relative to a front view of a patient;
transferring the determined anterior origin position of the transverse plane to a computerized image of the patient's head;
clinically determining the position of the posterior origin of the sagittal plane relative to a front view of a patient; and
transferring the determined posterior origin position of the transverse plane to a computerized image of the patient's head.

2. The method of claim 1, further comprising measuring soft and hard tissue landmarks to the three anatomical planes to define skeletal and soft tissue measurements of the face from a three dimensional view.

3. The method of claim 1, wherein the patient places his or her head in the natural head position and establishes the frontal postural head position by looking into a mirror at his or her pupils, leveling his or her head up and down along a sagittal plane and moving it side to side along a transverse plane in order to orient the frontal posture vertically and from left to right.

4. The method of claim 1, wherein the step of clinically determining the position of the coronal plane relative to a profile view of the patient's head comprises using a vertical line that passes through a point at subnasle and measuring the distance from vertical line to glabella.

5. The method of claim 1, wherein a cone beam computed tomography machine is used taking a multiple images of a patient's head and face in order to generate a 3-D computerized image of the patient's face and head, and wherein the 3D image generated is displayed in 3D software.

6. The method of claim 1, wherein the step of transferring the determined position of the coronal plane to the computerized image of the patient's head comprising displaying a computerized profile image of the patient's head, placing a mark that is the clinically determined distance from the coronal plane to the glabella, and tilting the image of the head as necessary until a vertical line drawn in software will run through the mark and through a point drawn at subnasle.

7. The method of claim 1, wherein the step of clinically determining the position of the transverse plane relative to a front view of a patient comprises clinically measuring hard tissue landmarks that generally correspond with the transverse plane and making any adjustments necessary to compensate for lack of symmetry of the hard tissue landmarks.

8. The method of claim 7, wherein the hard tissue landmarks comprise the patient's upper canine teeth.

9. The method of claim 1, wherein the step of transferring the determined position of the transverse plane to a computerized image of the patient's head comprises displaying a computerized front image of the patient's head, drawing a horizontal line on the computerized image of the patient's face through the hard tissue landmarks and tilting the image side to side in order to make any adjustments necessary to correlate the horizontal line with the clinically determined position of the horizontal line.

10. The method of claim 9, wherein in order to confirm the proper determined position of the transverse plane, the position of soft tissue landmarks and/or other hard tissue will be evaluated against the determined position of the transverse plane, and if discrepancies are detected, redoing the step of clinically determining the position of the transverse plane.

11. The method of claim 1, wherein the step of clinically determining the position of the anterior origin of the sagittal plane relative to a front view of a patient comprises taking a clinical measurement of the upper midpoint of the upper lip or some other point that is midpoint in the patient's face, and wherein the step of transferring the determined anterior origin position of the transverse plane to a computerized image of the patient's head comprises drawing a vertical line on the front view of the computerized image of the patient's head, and shifting the image of the head side to side until it is properly aligned with other landmarks.

12. The method of claim 11, wherein as a secondary check of the determined position of the anterior origin of sagittal plane, other landmarks are used selected from one or more of the nasal bridge and nose, and if the determined position of the anterior origin of sagittal plane is incorrect, retaking clinical measurements to determine the anterior origin of the sagittal plane.

13. The method of claim 1, wherein the step of clinically determining the position of the posterior origin of the sagittal plane relative to a front view of a patient comprises taking a clinical measurement of the left to right symmetry of maxillary second molars and noting any adjustments necessary, and wherein the step of transferring the determined posterior origin position of the sagittal plane to a computerized image of the patient's head comprises turning the image of the head from side to side so that the when an image of the underside of the patient's upper teeth is viewed, the sagittal line will pass midpoint between the maxillary second molars with any adjustments needed.

14. The method of claim 13, wherein as a secondary check of the determined position of the posterior origin of sagittal plane, other landmarks selected from one or more of the lateral orbital rims and mandibular angles are used, and if the determined position of the anterior origin of sagittal plane is incorrect, retaking clinical measurements to determine the anterior origin of the sagittal plane.

15. The method of claim 1, wherein the sagittal plane that comprises an anatomical midline of the patient's face has a projection that passes through the midpoint of the intercanthal distance (ICD-M) on the bridge of the nose and the midpoint of the upper lip as defined by the center of the philtrum (PH-M) and the upper lip cupid's bow.

16. The method of claim 1, wherein the sagittal plane is used to measure the distance to same landmarks on both the left and right sides of the face to determine if these landmarks are on the same level and/or are perpendicular to the anatomical sagittal plane to make a measurement of vertical symmetry.

17. The method of claim 16, wherein the landmarks on both the left and right sides of the face are selected from the group consisting of pupils of the eyes, maxillary canine teeth, mandibular canine teeth, and inferior border of the chin.

18. The method of claim 1, wherein the sagittal, coronal, and transverse planes are used to orient the face for measurement of the height and width and proportionality of those distances.

19. The method of claim 1, wherein the determined sagittal, coronal, and transverse planes are used to orient the face for vertical measurement of the face.

20. The method of claim 19, wherein the vertical measurement of the face are selected from the group consisting of the upper ⅓ height, lower ⅓ height, upper lip length, lower lip length, upper incisor exposure, upper incisor crown height, interlabial gap, upper vermillion height, and lower vermillion height.

21. The method of claim 1, wherein the sagittal, coronal, and transverse planes are used to orient the face for structural measurements selected from the group consisting of alar base width and maxillary intercanine width.

22. The method of claim 1, wherein the sagittal, coronal, and transverse planes are used to orient the face for shape and contour measurements of the cheekbone and the bridge of nose line.

23. The method of claim 1, wherein key structures of the face and head are measured to the sagittal, coronal, and transverse planes are selected from at least one of the group consisting of:
a. at least one of hard and soft tissue structures;
b. vertical structures;
c. outline structures;
d. structure levels;
e. midline structures;
f. left and right sides of face;
g. shape and contour symmetry; and
h. projections of facial landmarks.

24. The method of claim 23, wherein the at least one of hard and soft tissue structures include soft tissue and skeletal angles of the mandible.

25. The method of claim 23 wherein the vertical structures are selected from the group consisting of upper ⅓ height, lower ⅓ height, upper lip length, lower lip length, upper incisor exposure, upper incisor crown height, interlabial gap, upper vermillion height, and lower vermillion height.

26. The method of claim 23, wherein the outline structures are selected from the group consisting of left and right chin, mandibular angles, zygomatic arch width and mandibular body width.

27. The method of claim 23, wherein the structure levels are selected from the group consisting of eyes, maxillary canines, mandibular canines, commissures of the mouth, inferior border of the mandible and chin.

28. The method of claim 23, wherein the midlines structures are selected from the group consisting of the nasal bridge center, the nasal tip, the philtrum; the midline between the two central mandible incisors, subnasale and the midline of the chin and the midline structures are measured to the anatomical sagittal plane as defined inner canthal midline and philtrum midline.

29. The method of claim 23, wherein the left and right sides of face are selected from the group consisting of cheekbone height of contour and angles of the mandible.

30. The method of claim 23, wherein the shape and contour symmetry are selected from the group consisting of base of nose width and cheekbone contour.

31. The method of claim 1, wherein the sagittal, coronal, and transverse planes are used to orient key the face for measurement of internal structures including bone and teeth.

32. The method of claim 31, wherein the internal structures are selected from the group consisting of the piriform aperture, zygomatic arches, lateral orbital rims, zygomatic buttresses, angles of the mandible, body of the mandible, and parasymphasis of the mandible.

33. The method of claim 23, wherein after making measurements of the key structures of the face and head, frontal soft tissue and hard tissue facial analysis landmarks are identified using the anatomic frontal true vertical line.

34. A system and method for determining the correct natural head position locations of the coronal, transverse, and sagittal planes, relative to a three-dimensional computerized image of a patient's head, comprising, in not necessary the following order:
having a patient place his or her head in a natural head position;
clinically determining the position of the coronal plane relative to a profile view of the patient's head;
taking a multiple images of a patient's head and face in order to generate a 3-D computerized image of the patient's face and head and displaying the image in software;
transferring the determined position of the coronal plane to the computerized image of the patient's head;
clinically determining the position of the transverse plane relative to a front view of a patient;
transferring the determined position of the transverse plane to a computerized image of the patient's head;
clinically determining the position of the sagittal plane relative to a front view of a patient;
transferring the determined position of the transverse plane to a computerized image of the patient's head.
